(19) United States
(12) Patent Application Publication
(10) Pub. No.: US 2004/0225200 A1
(43) Pub. Date: Nov. 11, 2004

(54) System and method of analyzing the health of a population

(76) Inventors: Catherine M. Edmundson, Dunlap, IL (US); Linda G. Lavaux, Washington, IL (US); Richard C. Luetkemeyer, Peoria, IL (US); John C. Ruthman, Dunlap, IL (US); Syamala Srinivasan, Peoria, IL (US); Michael L. Taylor, Morton, IL (US)

Correspondence Address: CATERPILLAR INC., 100 N.E. ADAMS STREET, PATENT DEPT., PEORIA, IL, 616296490

(21) Appl. No.: 10/435,689

(22) Filed: May 9, 2003

(51) Int. Cl. A61B 5/00; G06F 17/60

(52) U.S. Cl. 600/300; 128/923; 705/2

(57) ABSTRACT

The present invention is associated with a system and method of analyzing the health of a population. The population includes a plurality of members. In one embodiment, the method includes the steps of establishing a plurality of health related characteristics associated with the population, the characteristics including a plurality of primordial characteristics, establishing a relationship between the health related characteristics and at least one health condition, and analyzing at least a portion of the population in response to the relationship. The analysis may include establishing at least one primordial characteristic associated with a health condition, applying an intervention in response to the characteristic, monitoring a success characteristic of the intervention, and determining a cause of the success characteristic

### TECHNICAL FIELD

This invention relates generally to a method and system of predicting a likelihood of developing a health condition, and more particularly to a method and system of predicting a likelihood of developing a health condition in response to primordial characteristics of the condition.

### BACKGROUND

Effective management of health care programs is difficult. Health care cost for health conditions continues to increase. Understanding how to apply finite resources in the most cost effective manner while supplying necessary health services is at best difficult based on known health care systems. In addition the root causes of health conditions are often not known. Therefore, managing care for health conditions based on unknown health causes increases the difficulty in achieving effective health care management. Some systems exist today, such as the system disclosed in U.S. Pat. No. 5,976,082, that disclose a system for predicting a future risk towards patients having an existing health condition. That is, once a patient has a health condition, the future risks of complications due to the existing health condition may be predicted. The system disclosed in 5,976,082 (the '082 system) does not look at the pre-disease state to predict the occurrence of the disease, but rather looks at the disease state to make predictions regarding future health issues based on the known disease. If a member does not yet have the disease, the disclosed '082 system will not help them. In addition, the system does not solve the problem of identifying the primordial characteristics of a health condition, such that future incidents of the health condition may be predicted, i.e., before the health condition occurs. Stated another way, the '082 system is directed towards traditional disease management as opposed to disease prevention. In addition, the type of information analyzed in the disclosure is limited to medical and drug claims.

The present disclosure is directed towards solving one or more of the problems set forth above.

### SUMMARY OF THE INVENTION

In one aspect of the present invention, a method of analyzing the health of a population is disclosed. The population includes a plurality of members. The method includes the steps of establishing a plurality of health related characteristics associated with the population, the characteristics including a plurality of primordial characterstics, establishing a relationship between the health related characteristics and at least one health condition, and analyzing at least a portion of said population in response to the relationship.

In another aspect of the present invention, a method of analyzing the health of a population, the population having a plurality of members is disclosed. The method includes the steps of establishing at least one primordial characteristic associated with a health condition, applying an intervention in response to the characteristic, monitoring a success characteristic of the intervention, and determining a cause of the success characteristic.

In yet another embodiment of the present invention, a system configured to analyze the health of a population is disclosed. The system includes a repository of health related characteristics associated with said population, the characteristics including a plurality of primordial characteristics; and a controller configured to receive the health related characteristics, establish a relationship between the health related characteristics and at least one health condition, and analyzing at least a portion of the population in response to said relationship.

### BRIEF DESCRIPTION OF THE DRAWINGS

FIG. 1 is an illustration of one embodiment of a method of analyzing the health of a population; and

FIG. 2 is an illustration of one embodiment of a system configured to analyze the health of a population.

### DETAILED DESCRIPTION

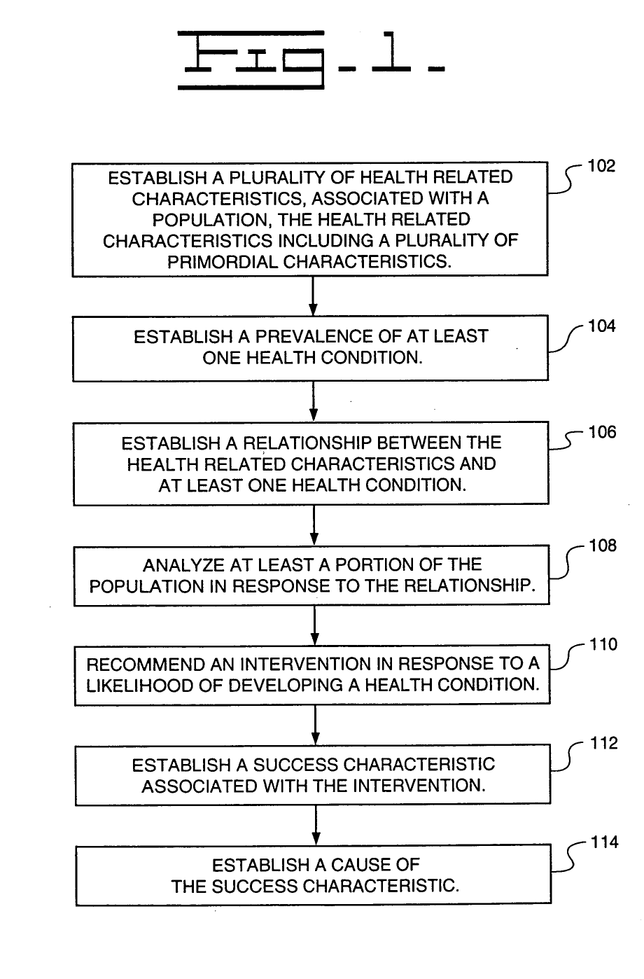

The present disclosure includes a system and method of analyzing the health of a population having multiple members. A population, as used herein, is any group of members. The population may include a high level of members, for example a group including one or more of the five kingdoms of living things, or a subgroup, for example a group including humans of a certain age range. The population may include living and/or dead members. As illustrated in FIG. 1, one embodiment of the method includes the steps of establishing a plurality of health related characteristics associated with the population, the characteristics including a plurality of primordial characteristics, establishing a relationship between the health related characteristics and a health condition, and analyze at least a portion of said population in response to the relationship. For example, the analysis may include predicting a likelihood of a member developing the health condition, in response to the relationship. The health condition may be any type of physical or mental health condition, disease, and/or ailment. In addition, the analysis may include predicting the incidence of the health condition. The analysis may also include performing a simple yes/no prediction regarding whether a member will likely develop the health condition.

The population associated with the analysis may be established by establishing the environment in which the analysis is to be performed. For example, the analysis may be used to enable the management of a health care program, such as a program associated with a corporation, or a program offered to the public by a health care consultant or provider. If the analysis is associated with a corporation's health care program, the population may include some or all of the employees and retirees of the corporation, and associated spouses and dependents. The population may include other associated groups of the corporation, such as consultants, contractors, suppliers and/or dealers. The population may include participants from multiple corporations and/or the general public. If the health care program is offered to the public, the population may include members of the public, organizations, and/or corporations.

In one embodiment of the present disclosure, in a first control block 102, a plurality of health related characteristics associated with the population, or a portion thereof, are established. The health related characteristics may include a plurality of health characteristics, lifestyle characteristics and/or family health characteristics associated with the members of the population. Health characteristics may include characteristics indicative of a specific member's health. For example, health characteristic may include medical characteristics (e.g., what medical visits, processes, procedures, or test have been performed associated with the member, the number of days the member has spent in a medical facility (e.g., a hospital), the number of visits the person has made to a doctor, etc.), drug characteristics (e.g., what type and amount of drugs are being consumed), a death characteristic (e.g., information associated with a death certificate), an absenteeism characteristic, disability characteristics, characteristics associated with existing health conditions, etc.

Family health characteristics associated with the member may include information associated with the family medical history of a specific member. For example, a history of a particular health risk within the family, e.g., heart failure, cancer, high blood pressure, diabetes, anxiety, stress, etc.

Lifestyle characteristic may include a specific member's behavior characteristic(s), of which some or all may be modifiable lifestyle characteristics. A modifiable lifestyle characteristic may include an exercise characteristic (e.g., does the member exercise, how often, what is the exercise, etc.) and/or a nutrition characteristic (e.g., what types of food does the member eat, and how often). Nutrition characteristics may also include the amount of salt consumed during a designated period (e.g., a day), and the amount of fat and/or saturated fat consumed during a designated period. In addition, modifiable lifestyle characteristics may include whether the member drinks alcohol (and if so how much), a drug intake characteristic, (i.e., does the member take drugs, and if so how often, what kind, and how much), a weight characteristic (e.g., what does the member weigh, what is the member's desired weight, is the member on a diet, what is the member's weight indicator e.g., obese, slightly overweight, underweight, normal, etc.), a smoking characteristic (does the member smoke and if so how much), a safety characteristic (what are the member's driving characteristics e.g., does the member where seat belts, have one or more infractions associated with driving under the influence, or speeding tickets, etc.). In addition, modifiable lifestyle characteristics may include a hypertension characteristic, a stress characteristic, a self-care characteristic, a self-efficacy characteristic, a readiness to change characteristics, and a prophylactic aspirin therapy characteristic.

In one embodiment, the health related characteristics may also include one or more of the following: demographic characteristics, the members location or geography, age, gender, employment status, employment type, and/or work characteristics of the member._The health related characteristics may be obtained through one or more of several sources, such as medical claims, drug claims, and/or self-reported characteristics (or data). In one embodiment, self-reported characteristics may be collected from the population. The amount and type of self-reported characteristics collected associated with the population is implementation dependent and may vary based upon the participation of the population, the relevance of the information to the different members of the population, and the analysis to be performed. Therefore the self-reported characteristics established may be associated with a subset, or portion, of the established population, or the entire population. The self-reported characteristics may include one or more health characteristics, family health characteristics, and lifestyle characteristics associated with a member of the population. The self-reported characteristics, also referred to as self-assessments, may be obtained through the use of one or more health related questionnaires submitted to the member. Examples of questionnaires include physical questionnaires, electronic questionnaires (e.g., located on a health related web-site), questionnaires filled out during a phone or personal interview, etc. The responses to the questionnaires may include a member's self assessed health related characteristics. The characteristics may include a self-efficacy characteristic and/or a readiness to change characteristic. A readiness to change characteristic is a characteristic indicative of a members readiness to change one or more behaviors, activities, or characteristics. A self-efficacy characteristic, as will be discussed, includes an indication of a member's belief in their ability to succeed in changing a lifestyle characteristic. For example the self-assessment questionnaire may specifically ask the member: does the member believe they can change their lifestyle or a specific aspect of their lifestyle, is the member willing to attempt to change an aspect of their lifestyle and if so, how successful do they think they will be, how important do they think it is to change one or more specified lifestyles, etc. Alternatively, one or more questions may be asked of which the answers may provide indirect indicators of whether the person actually does believe they can change aspects of their lifestyle, and also whether the member is actually ready to change a particular aspect of their lifestyle.

In one embodiment, the health related characteristics of the population are associated with self-reported biometric characteristics. For example, the sources of the health related characteristics may be self-reported biometric sources. That is, the sources of the health related characteristics are sources other than the direct physical examination of a member (e.g., where members provide a biological sample etc). The distinction is based on the issue that due to the size of the population, it may not be possible to analyze all of the members by having detailed examinations (e.g., blood samples, urine samples, etc.) of all, or even a substantial portion of the population. Therefore, in one embodiment, the health analysis is based on information that is obtained second-hand, without having physically examined a specific member to directly obtain the desired health related characteristics. Of course, if the described analysis indicates a particular member needs to be physically examined based on likelihood of occurrence of a health condition, that examination may occur. In one embodiment, the health related characteristics may related to non-intrusive characteristics, i.e., characteristics that do not directly involve the physical examination or taking of biological samples of a member by a physician. For example, a blood sample may be considered intrusive data because it involves the taking of a sample from a member.

The collected health related characteristics may be stored in a repository. The duration of storage is implementation dependent, but in general the more information available for analysis, the more accurate the results will be. Therefore, a historical repository of five to ten years may be established. In some embodiments, characteristics may be available throughout the working career of the members, e.g., if their employer collects self-reported information, medical and/or drug claims. The historical repository aids analysis in several ways, including reducing the impact of recall bias. Recall bias is what may happen when a member acquires a particular health condition, and then attempts to recall what factors may have contributed to the condition. The members recollection may be biased by any number of issues including their ability to accurately remember all the desired information. Therefore a historical repository aids in providing accurate information for analysis.

The established health related characteristics may be used to analyze the health of the population. In one embodiment, as illustrated in a second control block 104, a prevalence of a health condition within the population may be established. The prevalence of a condition may be described as the current existence of a condition. The prevalence of a health condition among a population may be described as the number or percentage of members that have a specific health condition. Establishing the prevalence of a condition in a population may include determining which members currently have a specific health condition. The prevalence of a health condition may be established by analyzing the health related characteristics associated with one or more members of the population and responsively establishing whether one or more members has the condition. For example, the prevalence may be established by analyzing information associated with medical claims and/or drug claims associated with the population.

Medical claims may include any type of health related correspondence between a health analyst or provider (e.g., doctor, physician, medical laboratory, hospital, medical support group such as x-ray providers, etc.), and a member of the population and/or a health care insurer, provider, or manager, for the member (e.g., corporation (employer) or third party insurer/manager, etc.). In one embodiment, the health related correspondence may include health codes such as E/M (Evaluation and Management) codes, Current Procedural Terminology (CPT) codes, and International Classification of Diseases (ICD) codes. ICD codes provide coded information associated with the treatment, health, and/or a condition of a member. These codes may include information associated with the professional services performed, the specific procedure(s) performed, and why the procedure(s) was performed. Therefore, analysis of ICD, CPT, and/or E/M codes may be used to establish whether a member has a particular health condition.

A drug claim may include any type of medication related correspondence between a medication provider (e.g., doctor, pharmacist, etc.), and a member of the population and/or a health care insurer, provider, or manager for the member (e.g., corporation (employer) or third party insurer/manager, etc.). In one embodiment, the correspondence may include codes or identification systems such as Group Product Index (GPI). The GPI provides a numbering system associated with the medication a member receives, and/or medication prescribed for a member. The GPI enables the identification of the type of drug, manufacturer, strength, associated dosage, and associated medication form (e.g., pill, tablet, liquid, etc.).

In one embodiment, information associated with at least one medical and/or drug claim may be used to determine the prevalence of a condition, e.g., whether a member has one or more specific health conditions. For example, if a medical claim indicates a particular procedure has been performed, then that procedure may be correlated to one or more potential health conditions associated with that procedure. Analogously, if a drug claim indicates that a member is being prescribed and/or receiving a particular medication, then that medication may be correlated to one or more potential health conditions associated with that medication. In this manner, the information associated with the medical and/or drug claims may be analyzed to establish a prevalence of a condition. The analysis may be performed on each member of the population, or a subset thereof.

In one embodiment, information associated with multiple medical and/or drug claims may be analyzed based on established criteria, to establish the prevalence of a health condition. For example, multiple medical and/or drug claims may be cross checked with each other to establish the prevalence of a health condition. An individual medical or drug claim may contain erroneous or misleading information. For example, there may be instances where a medical procedure is performed to test for a health condition, without definitively establishing the condition exists in the member. Analysis of the resulting medical claim may lead someone to erroneously believe the person had the health condition (e.g., based on the types of procedures being performed). Therefore, using one medical or drug claim may not provide an accurate indication of the presence of a health condition. Additional medical claims and/or drug claims may be analyzed to establish one or more health related characteristics of a member, such as the prevalence of a health condition. In one embodiment, multiple medical and/or drug claims, separated by a time period (e.g., a minimum duration) may be analyzed. The separation in time increases the confidence level regarding the determination that a particular health claim, or health related characteristic, exists. For example, two claims of the same type (e.g., two medical claims or two drug claims), separated in time by at least three months, may be analyzed to determine if a member has a health condition. If the first claim indicates a condition exists, and a second claim indicates the same condition exists, then the member may be assumed to have the condition associated with the medical claims. The two claims may sequentially occur, or be separated by one or more other medical and/or drug claims. In addition, a claim of one type (e.g., medical claim) may be cross checked with a claim of another type (e.g., drug claim). If the two claims correlate, then the member may be considered to have the particular condition. The two different types of claims may also be separated by a designated time period, e.g., three months, to further establish that the condition actually exists. In one embodiment, the time separation is established such that the two claims represent independent indicators, as opposed to two claims associated with the same medical event (medical checkup or medication collection). Additional criteria may include that the claims being correlated should occur within a particular time period of each other. For example, if two claims indicating a particular health condition are separated by five years in time, there is a chance that the claims were inaccurate anomalies as opposed to indications of the existence of the health condition. Therefore, a maximum duration between claims being cross checked may be established (e.g., one year). In one embodiment, the maximum duration between cross checked claims may be dependent upon the condition at issue. For example, some health conditions may be more likely to have multiple claims occur within a specific time duration. While other health conditions may not manifest themselves in multiple medical claims in that same specified time duration. Therefore, the duration between claims may be implementation and health condition dependent.

In one embodiment, a look up table may be used to cross check the claims and/or associate the claims with one or more health conditions. When claims are received, they may be manually or automatically analyzed. For example, when a claim is received, it may be analyzed to establish associated health characteristics. The health characteristics may then be cross checked with information from other claims in an attempt to verify one or more of the health characteristics. The analysis may include correlating the claim with a table of potential health characteristics associated with claim information. The information may be compared with previous claim information to determine if prior claims indicated the same, or similar health related characteristics. If the cross check indicates one or more prior claims indicated the same health related characteristic, then the member may be assumed to have the health related characteristic (e.g., the health condition). If no prior claim information correlates with the current claim information, then the current claim information, and the correlated health related characteristics may be store to be compared with future claims that are to be receive. In one embodiment, if a strong correlation exists between the health related characteristics associated with multiple claims, and a sufficient time period exists between the claims, then the member may be determined to have the characteristics. Alternatively, statistical analysis techniques such as classical, Bayesian, and/or machine learning analysis techniques may be used to correlate and cross check one or more medical and/or drug claims with one or more health related characteristics and/or health conditions. For example, neural networks may be trained to associate information associated with medical and/or drug claims with particular health related characteristics and/or health condition. Then when a claim is received, it may be analyzed to establish potential health related characteristics and/or health conditions. The neural network may be able to provide a weighted analysis such that the results have an associated confidence factor. If multiple claims separated in time indicate the same or similar health related characteristics, the resulting neural network analysis may provide a higher confidence indicator than if just one claim indicated the characteristics. Therefore, as prevalent health conditions are established based on medical and/or drug claims, the claims may be further analyzed to establish a relationship capable of automatically detecting a prevalence based on the available medical and/or drug claims.

In one embodiment, the medical and/or drug claims may be analyzed as they are received. Alternatively, there may be a repository of one or more previous medical and/or drug claims associated with the member(s). For example, repositories may be created that include a members historical health related characteristics over a time period (e.g., the last five-ten years). These repositories may be maintained by the health care provider, insurer, analyzer, and/or manager. These repositories may be analyzed to establish a prevalence of a condition among the population.

In one embodiment, self-reported characteristics may be analyzed to establish the prevalence of a condition, e.g., among one or more members of a population and/or the population as a whole. For example, a member may specifically indicate that they have a particular condition. Alternatively the analysis of one or more of the family history, lifestyle, or health characteristics indicated through the self assessments may indicate that the member likely has a particular condition. In this case additional follow-up may be performed with the member to determine if they actually have the condition, or know that they have the condition. In one embodiment, a relationship may be established to determine the existence of a condition among a particular member and/or among the population.

In one embodiment, medical claims, drug claims, and self-reported characteristics may be used to establish the prevalence of a particular condition among the population. Alternatively, as indicated above, the prevelance of a condition may be established based on one or more of the sources of information (e.g., medical claims, drug claims, and/or self-reported characteristics). The prevalence may be established manually or through an automated process such as the use of statistical analysis techniques as mentioned above. The decision of what information (or sources of information) to use may be based on what information is available for the population, or for a particular portion of the population. For example, some portions of the population may not have a historical data base of information available for analysis. In addition, some portions of the population may not have associated medical claims, drug claims and/or self-reported characteristics. Therefore the type and amount of information to be analyzed to establish the prevalence of a disease is implementation dependent and may be based in part on the type of information available for a particular population, or portion thereof.

Referring again to FIG. 1, in a third control block 106, a relationship may be established between the health related characteristics and one or more health conditions. In one embodiment, the relationship is established in response to the prevalence of the health condition. The relationship may then be modified based on future occurrences, or rates of occurences of the condition. Alternatively, the relationship may be established by analyzing future occurrences or rates of occurence of the condition without accounting for an initial prevalence of the condition. In one embodiment, as described below, the relationship may be used to predict an incident or occurence of a disease, e.g., an occurence of a disease among a particular member or among a population in general. Information gained from establishing the prevalence of the condition may be used to establish the relationship. For example, the health related characteristics associated with the members determined to have a particular condition may be analyzed to establish a relationship associated with a likelihood of developing the condition. In one embodiment, the predictive relationship is different than the analysis to determine the prevalence of a condition because the prevalence analysis may be used to establish who has the condition. However, the occurrence predictor may be used to establish the health related characteristics that are needed to predict the likelihood of developing the health condition. In one embodiment, all of the health related information associated with a member having a condition, or all of the health related information believed to be potentially relevant to a health condition that a member has, may be analyzed to establish the relationship. The health related information to be analyzed may be historical data that pre-dates the incidence of the health condition. For example, if a member is determined to have a particular health condition on Oct. 1^{st}, 2003, then the data to be analyzed may pre-date October 1^{st}. The historical data may go back five years or more in time.

The analysis and associated relationship may indicate the primordial characteristics associated with a health condition. Primordial characteristics are characteristics that impact the chance of acquiring risk factors associated with a condition. A primordial characteristic may be associated with more than one risk factor and/or more than one health condition. A heart attack is an example of a health condition. Risk factors associated with a heart attack may include obesity, age, and gender. A risk factor may be described as one form of a heath related characteristic that is a known, believed, or hypothesized to be an indicator of acquiring a health condition, or increasing the risk of acquiring the health condition. Risk factors usually have one or more primordial characteristics associated with them. Primordial characteristics associated with obesity, or being overweight, include dietary characteristics, such as the amount of saturated fat, fiber, and calories consumed during a time period. By reducing the amount of saturated fat consumed (a primordial characteristic), the chances of acquiring the associated risk factor may be reduced (e.g., reduced chance of being overweight). If the chances of acquiring a risk factor is reduced or eliminated, then the chances of acquiring an associated health condition (e.g., the heart attack) are also reduced or possibly eliminated. As will be discussed, one embodiment of the present disclosure is associated with identifying primordial characteristics associated with a health condition, and then predicting an incident of the condition associated with a particular member based on the particular health related characteristics associated with that member. Intervention recommendations may then be tailored to the particular primordial characteristics exhibited by the particular member. In one embodiment, the collected health related characteristics may be detailed and extensive in order to acquire the desired information that may be associated with possible primordial characteristics. By the nature of the analysis being performed, the primordial characteristics may not be initially known. Therefore, monitoring of future occurrences of the condition, and analysis of the associated health related characteristics enables the predictive relationship to evolve as new information is available.

The details of the establishment of the relationship will be described below. However, in general, the health related information associated with the population will be analyzed to establish the relationship. The analysis may include the use of statistical analysis techniques such as classical, Bayesian, and/or machine learning analysis techniques to analyze the health related information. For example, neural networks may be trained using all the health related characteristics of the members having a particular condition. Then, the health related characteristics of a member of the population may be delivered to the neural network for analysis. The resulting analysis may provide a weighted answer indicative of the likelihood the person will acquire the condition. In addition, review of the neural network may provide insight into which health characteristics are more relevant to acquiring the condition. These characteristics may then be reviewed to establish the primordial characteristics associated with the condition. For example, the relevant health related characteristic may be a primordial characteristic, or may have associated primordial characteristics. Therefore, depending on the specific implementation used, the analysis may be able to indicate the health related characteristics that are most relevant to the prediction of a particular health condition.

In a fourth control block 108, the relationship may be used to analyze the population with respect to the health condition. For example, the relationship may be used to predict the likelihood of developing a condition associated with the population, or a portion thereof. As such, the relationship may be used to predict a future incident or occurrence of the health condition. In one embodiment, the health related information associated with one of the members of the population may be analyzed using the established relationship. The analysis may indicate, or predict, whether the member will develop a particular disease, which may include the likelihood the member will develop the particular disease. The analysis may be used to predict the occurrence of the disease based upon the established primordial characteristics. In addition, this analysis, or information resulting from the analysis may be used to predict an incidence of the health condition, e.g., over a specified period of time, how many members will develop the disease, or what is the likelihood of a particular portion of the population developing the disease over a specified time period.

In one embodiment, depending on the health condition associated with the analysis, the analysis may also establish a predicted time period in which the incident may occur (e.g, the next year, next five years, next ten years, etc.). In addition, the analysis may establish a stage of the condition associated with a particular member. For example, some conditions may have definable stages of the onset of the disease.

In one embodiment, a likelihood of developing the condition may be established based upon the analysis. For example, some analytic techniques produce information associated with the likelihood of having the incidence, e.g, a confidence level. Therefore, the analysis may include classifying all or a portion of the population with respect to one or more conditions, based on the likelihood of the particular members having an incidence of the disease, based on the particular stages of the condition the population members fall within, and/or based on the predicted time period associated with the incidence. In this manner the population may be classified, or ranked, with respect to the likelihood of developing a condition, the time period in which the development may occur, and/or the stage of the condition the member is in. As described below, this classification, or ranking of the population, or a portion thereof, with respect to one or more conditions enables, specific interventions to be applied to specific members based on predicted risk, and also enables the management of the population as a whole, and the intervention and associated cost, etc. Therefore, the population may be analyzed to establish a likelihood of developing of one or more conditions, among one or more members of the population.

In one embodiment, as illustrated in a fifth control block 110 in FIG. 1, an intervention may be recommended in response to the likelihood of developing the health condition. Factors that may be used to select the appropriate intervention include the predicted likelihood the member will develop the health condition and the primordial characteristics the member exhibits that are associated with the condition. For example, the more likely the person is to acquire a particular condition, the more aggressive the intervention recommendation may be. Other health related characteristics may also be used to determine the appropriate intervention, such as the self-efficacy characteristic and/or readiness to change characteristic associated with the member, and the likelihood of success of the intervention. The cost of the intervention may also be a factor in intervention selection. The role of intervention cost may be based on the premise that there is a finite amount of money available to administer health care interventions to the population. Therefore, one use of the analysis may be to determine how the interventions may be applied in a cost effective manner, while providing the best benefit for the population. For example, given the choice between recommending an intervention that is 60% effective and an intervention that cost twice as much, but is only 62% effective, the decision may be to apply the less expensive, yet effective intervention, and use the “savings” in other areas of the health care program.

In one embodiment, as illustrated in a sixth control block 112, the method may include the step of establishing a success characteristic associated with the intervention. The success characteristic may include a characteristic associated with the success of the intervention, e.g., did the intervention succeed (or assist in succeeding) the prevention or delay of the incidence of the health condition. The success characteristic may include characteristics associated with whether the intervention was used, to what degree the intervention was used, why the intervention was, or was not used, and how effective was the intervention in light of how much it was used. Some of these success characteristics may be established shortly after recommending the intervention (e.g., was the intervention used and why or why not, to what degree the intervention was used, if not used what would it take to motivate the member to use, etc.), while other success characteristics may not be established for a period of time (e.g., if used, how successful was the intervention).

Success characteristics may be collected in several ways. The characteristics may be collected through medical and/or drug related information. For example, an intervention may include a recommendation that the member visit a medical provider (e.g., doctor), have a medical test performed, and/or be prescribed a particular drug. The members medical and/or drug claims may be monitored to determine if the recommendation was followed. For example, if over a particular time period, e.g., three months, there is no indication from reviewing medical claims, that the member visited a medical provider, then the assumption may be made that the member did not follow the recommendation. In light of this, a health care counselor or provider may be notified, and the member contacted to verify they did not follow the recommendation, and determine why the recommendation was not followed (if indeed it wasn't). This may be done by monitoring claims either manually or in an automated fashion, e.g., through the use of a computer program. For example, once an intervention is recommended, a computer related program may be configured to automatically review medical, drug claims, and/or self assessment characteristics to monitor characteristics of whether the recommended intervention was performed.

The success characteristics may be collected through self-reported data (e.g., targeted questionnaires, interviews, one on one phone calls such as counseling phone calls, etc.). For example, if a particular intervention recommended a medical visit, the targeted questionnaire or counseling call may specifically inquire as to whether the medical visit was made, and why or why wasn't the visit made.

In one embodiment, in a seventh control block 114, a cause of the success characteristic may be established. For example, the success characteristics, may include information indicative of a persons self-efficacy, and/or readiness to change. For example, if the established predictive relationship indicates that a particular member is at risk for a heart attack, and part of the primordial characteristics associated with a heart attack is that the member is eating too much saturated fat, an intervention recommendation may include recommending a change in lifestyle, e.g., increased exercise such as running, walking, or swimming. The success characteristics may indicate that the person did not engage in any exercise (e.g., on a subsequent self assessment). Upon further follow up (e.g., within the same questionnaire or counseling session, or in a later one), the member may indicate that they don't enjoy exercising and/or they don't perceive the need to do so. Alternatively, the member may indicate that they tried running, but their knees hurt, so they stopped, and/or they did not have access to a swimming pool. That is, while they are willing to engage in a recommended intervention, the specific intervention recommended did not work for them. Alternatively, the member may indicate that while they enjoy working out, they do not have time outside of their work and family activities to engage in the recommended intervention. The measured characteristics may also indicate that the recommended intervention was followed. In this case, information may be obtained regarding why the recommendation was followed.

The success characteristics may be used in several ways. In one embodiment, analysis may be performed with the success characteristics to establish a relationship capable of indicating or predicting a members engagement of an intervention, or willingness to engage in a particular intervention, or in any intervention. For example, the health related information, including the success characteristics, associated with members who have been recommended a particular intervention, may be analyzed. The analysis may result in a relationship that is able to establish the likelihood a particular member will follow a particular recommendation, based upon the specific health related information associated with the member. In one embodiment, the success characteristics may be used to establish a relationship capable of indicating or predicting a particular members engagement of any recommendation, or willingness to engage in any intervention. In one embodiment, the analysis may include establishing a relationship capable of predicting a members readiness to change stage. That is, in one embodiment, readiness to change categories may include a pre-contemplation, preparation, and action stages. If a member is in a pre-contemplation stage, they may not be willing to engage in any intervention. In the preparation stage, a member may be willing to pursue a particular intervention, but not just any intervention, or they may be willing to pursue interventions, but have not started. In the action stage, the member may be ready to take action in the appropriate intervention. By classifying a member into a readiness to change category, interventions may be further tailored for the individual member. For example, if a member is in the pre-contemplation stage, then the selected intervention may include additional counseling and/or educational literature associated with the seriousness of the potential condition, and the risk associated with this particular member of acquiring the condition if no action is taken. In addition, the intervention associated with the preparation category may include customizing the proposed intervention to the interventions the member is more likely to pursue. In this manner, self-efficacy and readiness to change characteristics associated with a particular intervention, may be analyzed with other self-efficacy and readiness to change characteristics associated with other interventions, and applied to the population as a whole where appropriate. That is, some established self-efficacy and readiness to change characteristics may be generalized (e.g., by creating a predictive relationship) and applied to the whole population to predict a particular members likelihood to engage in a particular intervention, or an any intervention. The analysis may include using statistical analysis (e.g., neural networks, regression analysis, etc.) to establish a relationship that is able to predict a members willingness or ability to pursue a particular intervention. In this manner a relationship may be developed and used in future instances such that when a member is predicted to have an incidence of a condition, the recommended intervention may be based upon indirect indicators of a members self-efficacy or readiness to change, as well as direct indicators (e.g., specific questions such as: are you willing to reduce your smoking).

For example, potential interventions may include an exercise regimen, a dietary regimen or a medication, to reduce the risk of a condition. The exercise regimen may indicate the highest success rate if followed, the medication the lowest success rate if followed. In addition, the member may provide strong direct indications of self-efficacy and readiness to change characteristics. However, the established participation predictive relationship may indicate that members with similar health characteristics (e.g., job requiring long hours, area of the country not conducive to exercise during the winter, and number of dependents in the family), that the member is not likely to follow through on an exercise regiment (e.g., due to time constraints from the job and family and inclimate weather). However, based on the other members, it may be predicted that this member is most likely to follow through a dietary change. Therefore, the intervention may be targeted to either changing the dietary habits of the individual.

In addition, the analysis of the success characteristics and associated health related characteristics may include establishing a relationship able to indicate a potential success of a particular intervention. For example, the success of an intervention may be established by monitoring/analying the health related information for an extended period of time. The health related information, including characteristics indicative of the incidence of the health condition may be monitored after the intervention is applied, and compared to health related characteristics expected if the intervention had not been applied (e.g., whether an incidence of the health condition would occur, when it would occur, when the stages of the incidence (if any) would occur. In addition, the health related characteristics may be analyzed to determine if any immediate changes in health care characteristics occurred. For example, if the health condition is a heart attack, and one of the primordial characteristics associated with heart attacks is a members consumption of high saturated fats, then one intervention recommendation may be a dietary program. The health care characteristics may be monitored to determine if the dietary program was successful in reducing the members saturated fat consumption, and/or whether the dietary program was successful in eliminating or delaying the incidence of the health condition. Therefore the success of the intervention may be monitored with respect to eliminating or delaying the health condition, and/or eliminating or reducing a cause associated with the health condition. The results of the intervention monitoring and associated health related characteristics may be used to further refine the decision process regarding which intervention to recommend.

The health related characteristics, including the success characteristics, may be used to select one or more interventions for a particular member at risk of a particular condition. In addition, the health related characteristics may be used to establish a relationship that associates one or more interventions with particular health related characteristics and a health condition. The success characteristics may indicate that interventions have varying degrees of success based upon the health related characteristics such as the physical characteristics of the individual engaged in the intervention, the thoroughness of the use of the intervention, the willingness of the person to pursue, etc. For example, assume there are two potential interventions for a health condition. Assume intervention 1, if followed 100%, is 90% effective, and if followed 50% is 30% effective. In addition, assume intervention 2, if followed 100% is 60% effective, and if followed 50% is 45% effective. Depending on the health related characteristics of a particular member, the best chance of preventing or delaying the condition (or eliminating a cause of the condition) may lie with pursuing intervention 2. For example, if the self-efficacy characteristic, or readiness to change characteristic is low, this may be an indication that the member won't follow through completely with the recommendation. Therefore, the second intervention may be pursued that may have a better impact than the first intervention given that neither intervention is used completely. Therefore, a relationship may be developed that is able to predict the effectiveness of a particular intervention in general, e.g., if the intervention is used X%, then it will be Y% effective. This information may be used to make an intervention recommendation to a member or, to engage in further correspondence (interviews, follow-up questionnaires, etc.) with the member. For example, the member may be notified of the preferred intervention, but of the concerns that they are not going to fully engage the intervention. If they don't fully engage the intervention then there is an alternative intervention that is preferred. In addition, a relationship may be established to predict the useage of an intervention by a particular member, based upon the health related characteristics of the member. The relationship may also be able to predict the success of a particular intervention based on the predicted use of a member (e.g., based on the members self-efficacy, readiness to change, and or other health related characteristics). In addition, interventions may have varying success among different members, even if pursued to the same degree. Therefore, the recommendations may be modified based on any previous engagement by the member in an activity related to an intervention. For example, health related characteristics associated with the member and the activity the member engaged in may be used to tailor the specific recommendation provided.

In one embodiment, the success characteristics associated with a particular member may be used to further refine, or establish, a recommended intervention for the member. In addition, the success characteristics may be used to refine the analysis (e.g., relationship) that correlates a member of a population with a particular intervention, based on the health related characteristics of the member.

Establishing the Relationships

As mentioned above, a relationship is established between the health related characteristics and a health condition. The type of analysis used to establish the relationship is implementation dependant and may vary as a function of the data available information being requested (e.g., explain the similarities/dissimilarities of the health related characteristics of members having the condition, predict future incidences, or both). The analysis may be dependent on the number of dependent variables (e.g., the health condition(s) associated with data) and/or independent variables (e.g., health related characteristics) that are being analyzed in the relationship and/or the objective of the analysis being performed. For example, the analysis may include the use of statistical analysis techniques such as classical, Bayesian, and/or machine learning techniques. Classical analysis techniques may include multivariate statistical techniques simple regression, multiple regression, factor analysis, item analysis multivariate analysis of variance, discriminant analysis, path analysis, cluster analysis, multidimensional scaling, rule induction, and/or least squares estimation. In one embodiment, multiple regression may be used to determine the relationship between one dependent variable (e.g., whether a person has diabetes) and multiple independent variables (i.e., multiple other health related characteristics, such as weight, gender, age, dietary habits, excercise, etc.). Other techniques, such as in factor analysis, cluster analysis, and multivariate techniques may be used when the desired relationship is associated with multiple dependent variables and multiple independent variables. Generic model-fitting or classification algorithms e.g., neural networks (e.g., back propagation, feed-forward networks, etc.), meta-leaming techniques such as boost, etc., may be applied for predictive data mining. Predictive data mining techniques may be desired when the accuracy of a prediction is of higher priority, regardless of whether or not the models or techniques used to generate the prediction is interpretable or open to simple explanation. That is, data mining techniques may be desired when the objective is to predict the future occurrence of a health condition, as opposed to analyze the existing relationship among the health related characteristics that leads to the health condition. As mentioned, the selection of the particular analysis technique(s) is implementation dependent and may be based on factors such as user preference, the data to analyze, and the number of dependent and/or independent variables, the objectives of the analysis. Therefore, in one embodiment of the present disclosure, the person analyzing the health of the population may specify the analysis techniques to be used, or the analysis system may automatically determine the appropriate technique(s) to use.

In one embodiment, data analysis using neural network techniques may be used to establish a predictive relationship between the health related characteristics and one or more health conditions. For example, the health related characteristics associated with members known to have a particular health condition, may be used to “train” the neural network. There are several types of neural network models. The selection of which model or combination to use may be implementation dependent, and implementation accuracy may vary based on model used, data analyzed, and desired objective of the model. In one embodiment, the model used is a back propagation network. The back propagation network may receive the health related characteristics associated with the members known to have a particular health condition, along with the characteristics of members known not to have the characteristic. The resulting “trained” neural network may then be able to receive the health related characteristics of a member to predict whether they will acquire the health condition. In one embodiment, the neural network output is a number (e.g., between zero and one), that may be used to indicate that the member has a determined likelihood of having an incidence of the condition (e.g., 75%), if they do not already have it. As was discussed above, the resulting likelihood of occurrence may be used to rank the population in terms of likelihood of acquiring the condition. This ranking may then be used to prioritize intervention strategies.

In addition to establishing a likelihood of occurrence of the health condition, the internal organization of the neural network may be analyzed to determine which health related characteristics where most relevant to the condition. For example, a back propagation network includes multiple weighted interconnections between the input factors and the output. The weighted interconnections may be reviewed and correlated with the input health related characteristics. In this manner, the characteristics having more relevance (e.g., a higher weighting value) may be identified. These relevant characteristics may then be reviewed to establish the primordial characteristics of associated with the health condition. For example, the established health related characteristics may already be primordial characteristics (e.g., the amount of salt consumed per day, the amount of saturated fat consumed per day). However, if the established health related characteristics are not primordial characteristics, then further analysis may be performed to break the characteristics into the primordial characteristics. For example, if being overweight is established as a relevant health related characteristic, then further analysis may be performed to determine what primordial characteristics lead to being overweight, and which of these primordial characteristics did members being analyzed exhibit. In one embodiment, all of the factors associated with being overweight may be treated as being relevant. Alternatively, the potential primordial characteristics are used to further refine the relationship to establish which of the primordial characteristics plays a role in being overweight, when overweight is a factor in having a particular health condition, e.g., diabetes.

In one embodiment, if multiple regression is the analysis technique used, an equation associated with the relationship may be: Y=b1X1+b2×2+ . . . bnXn+c, where the b's are the regression coefficients, representing the amount the dependent variable Y (e.g., likelihood of contracting a health condition) changes when the independent variable (the X's, e.g., the health related characteristics) change 1 unit. The c is the constant, where the regression line intercepts the y axis, representing the amount the dependent variable Y will be when all the independent variables are 0. In one embodiment, a determination may be made regarding whether any transformation (e.g., log functions, square roots, etc.) are needed to the proposed relationship (or equation). For example, should the log of a health related characteristic be used in the relationship, should the square root of a health related characteristic be used in the relationship, etc. As will be discussed, the form of the equation, e.g., whether one or more transformations are used, may be determined by the user, by the analysis system, or a combination thereof.

In one embodiment, different relationships may be created, e.g., using different transformations or different health related characteristics for the multiple regression analysis, and analyzed to determine which relationships perform better than others. Goodness of Fit analysis techniques such as R2, RMS, P Value, F ratios, standard error, etc., may be used to establish performance characteristics of the relationships. For example, techniques such as R², which establish the percent of variance in the dependent variable (e.g., the part characteristic cost), explained collectively by the independent variables (e.g. the other part characteristics). By using R², for example, an assessment may be made regarding which relationship best explains the variance in the dependent variable in response to the independent variables. RMS provides an indication of which model best predicts future aspects of a part, or part to be designed.

Therefore, in one embodiment, a threshold level of desired performance may be established for the relationship. If the relationship does not meet the threshold level of desired performance, then the user may be notified that the established relationship does not meet the desired level of accuracy, the desired level of ability to explain the variance in the dependent variable in response to the independent variables, or desired level of ability to predict future characteristics of the part. If multiple relationships are being compared with each other, and none of them exceed the desired level of success, then the user may be notified of which relationship performed best, but that none of them met the desired threshold. If multiple relationships are tested and one or more exceed the threshold, the best one may be selected, or they may all be provided to the user for selection.

In one embodiment, the analysis system may also establish which health related characteristics are more relevant to the relationship than others. In this embodiment, health related characteristics that are not significant to the relationship may be removed from the analysis. In one embodiment the relevance of the health related characteristics may be established by analyzing the established relationship. Therefore, a comparison of coefficients may be performed to establish the influence (or sensitivity), or relative influence of one health related characteristic versus another. In one embodiment, a system may utilize additional statistical analysis to establish the relevance of the independent variables and/or to select which variables to use in the relationship. Applicable techniques such as stepwise multiple regression (including forward selection, or backward elimination), forced entry, forced removal, and hierarchical multiple regression may be used. For example, multiple regression analysis may be used to establish a relationship between all of the independent variables (e.g., health related characteristics), and the dependent variable (the health condition). The relationship may establish a relative influence of the independent variables. Then, forward selection (associated with stepwise regression) may be used to determine the relevance of the variables. Forward selection may begin with no independent variables in the equation (associated with multiple regression). The independent variable having the highest correlation, or influence, with the dependent variable may be added into the equation. The performance of the resulting equation may be determined using the assessment techniques previously discussed, such as R². The process may be repeated, adding another independent variable (and associated coefficient) to the equation, and then assessing the equation. Once all the independent variables have been added, the assessment metrics (e.g., R²) may be compared to determine which equation best described the relationship. The variables in the equation that best describes the relationship may be considered to be the most relevant variables, and the other variables may be ignored. For example, a determination may be made regarding which variable (or characteristic) configuration resulted in the highest R², or noticeable improvements in R². Alternatively, each time an independent variable is added, the relationship is assessed to see if there was a noticeable improvement (e.g., was R² increased by an appreciable amount). If the assessment metric was not increased by a significant amount, then the process may be stopped, and the independent variables currently forming the relationship may be deemed to be the most relevant. The amount of increase in R² that triggers the completion of the process is implementation dependent.

The backward elimination process (associated with stepwise regression) begins with all the independent variables in the equation and sequentially removes them, analogous to the forward process, to determine the desired relationship. For example, after establishing the relative influence of the independent variables, the least influential independent variable may be removed from the equation. If the resulting R² is not significantly reduced, then the process may be repeated. In one embodiment, stepwise regression may be used when constructing the equation, or to prune the variables (or characteristics) used in establishing the equation. Therefore, the system is able to establish the health related characteristics relevant to the likelihood of contracting a particular health condition. The established health related characteristics may already be primordial characteristics (e.g., the amount of salt consumed per day, the amount of saturated fat consumed per day). However, if the established health related characteristics are not primordial characteristics, then further analysis may be performed to break the characteristics into the primordial characteristics.

In one embodiment, a system is configured to automatically form a relationship between the selected health related characteristics and the selected health condition (e.g., diabetes). The system may automatically develop multiple relationships, using different analysis techniques, and/or different transformations for the techniques, and/or different independent variables in the relationships. The results may then be compared to determine which relationship most accurately describes the relationship. For example, techniques such as R² may be used to establish, the accuracy of each relationship (or model). The resulting comparison may be used to select the most accurate relationship. In one embodiment, the system may step establish relationships based on all of the available techniques (e.g., a toolbox of Classical, Bayesian, and Machine learning techniques, and/or a combination thereof), and then compare the performance capabilities to establish the most appropriate relationship(s). Alternatively the system may select analysis techniques based on analysis characteristics, such as the number of dependent variables to be analyzed, the number of independent variables to be analyzed, the objective of the analysis, the type of data involved, and/or the class of problem at issue. As will be described, in one embodiment, the user may enter one or more of the above analysis characteristics. In one embodiment, multiple relationships may be established to support different analysis objectives, e.g., to analyze a relationship among current health related characteristics or predict future incidence of a health condition. Therefore, the user may enter the analysis technique(s) to use, the system may recommend analysis technique(s) to use based on user inputs, or the system may automatically establish the techniques to be used so the user doesn't have to select a desired technique and/or associated technique configuration information.

In one embodiment the system may also establish confidence factors associated with the relationship, or results of the relationship. For example, the relationship may indicate that there is a 70% likelihood that the member being analyzed will have an incidence of the condition.

Therefore, a relationship between the health related characteristics, and one or more health conditions may be established. As discussed above, the relationship may be used to predict an incidence of the condition among a particular member of the population based on the health related characteristics associated with the member. In one embodiment, the process and the algorithms used to develop the predictive relationship are re-useable, and may be used to establish other relationships associated with the health analysis process. For example, using analogous analysis techniques, a relationship may be established that will establish the prevalence of a health condition among one or more members of the population, as previously discussed. In addition, analogous analysis techniques may be used to establish a relationship that will provide an intervention recommendation for a particular member based on the health related characteristics of the member. In another embodiment, analogous techniques may be used to establish a relationship between a self-efficacy and/or a readiness to change of a member, and the health related characteristics of the member. Therefore, the established health related characteristics may be used to establish multiple relationships used for the analysis of the health of the population. In addition, an analysis tool may be developed that automatically develops the desired relationship based on the user defined independent variables (e.g., the health related characteristics), dependent variable(s) (e.g., the variables that are being pursued, such as likelihood of a condition incidence, recommended intervention, etc.), and/or objective of the user (e.g., to define and explain a relationship, to predict future incidences based on past occurrences, etc.). These parameters may be used by the system to automatically configure the desired relationship. Upon completion of the relationship, health related characteristics of a particular member may be delivered to the system to be analyzed by the relationship.

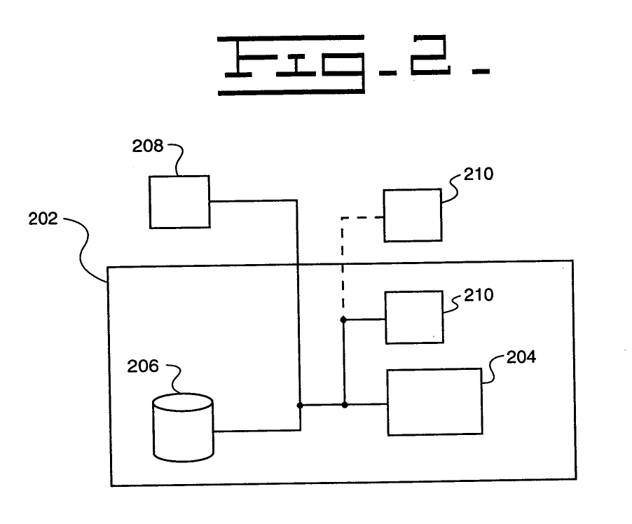

FIG. 2 illustrates one embodiment of a system 202 configured to analyze the health of the population. The system 202 is configured to perform the method illustrated in FIG. 1, or a portion thereof. For example, the system 202 may include a controller 204 configured to establish a plurality of health related characteristics associated with the population, establish a relationship between the health related characteristics and at least one health condition, and predict a likelihood of developing at least one health condition, associated with at least one of the members, in response to the relationship. The system 202 may include a repository 206 of health related characteristics associated with the population. In addition the system 202 may electronically receive health related characteristics from one or more remote computers 208. For example, medical and/or drug claims may be electronically (and in one embodiment automatically) delivered to the system 202 once they are generated by the remote computer 208. In addition, the system 202 may include a web-based interface 210 for members to interact with to provide self-assessments to the system 202.

In one embodiment, the controller 204 may periodically analyze the members of the population based upon the relationship and the health related characteristics associated with that member. Alterantively, whenever health related characteristics associated with a particular member are received, the system 202 may automatically analyze the likelihood of that member developing a health condition in response to the relationship, and the new and existing health related characteristics.

### INDUSTRIAL APPLICABILITY

The present invention includes a method and system configured to analyze the health of a population having multiple members. In one embodiment, the method includes the steps of establishing a plurality of health related characteristics associated with the population, the characteristics including a plurality of primordial characteristics, establishing a relationship between the health related characteristics and the health condition, and predicting an incident of the health condition associated with at least one of the members, in response to the relationship. The health condition may be any type of physical or mental health condition, disease, and/or ailment. For exemplary purposes the method and system will be discussed as they may relate to the health condition diabetes.

A repository of health related characteristics associated with a population may be collected. The health related characteristics may be collected through sources such as medical claims, drug claims, and self-reported information. The characteristics may include health characteristics, lifestyle characteristics, and family history characteristics. The characteristics may include the amount of saturated fat, unsaturated fat, fiber, salt, alcohol, cholesteral, etc. that a member consumes in a give time period. The characteristics may include weight characteristic, such as a members weight, BMI (what does BMI stand for), abdominal girth, etc. The characteristics may also include the persons blood pressure, standing heart rate, exercise habits (type and duration), and whether the member has hypertension.

The health related characteristics of the population may be analyzed to establish the prevalence of diabetes among the population. For example, any medical claim having an ICD code with the prefix 250 is an indicator that the member may have diabetes. In addition, drug claims having a medication code descriptive of an anti-diabetes medication are indicators that the member has diabetes. The medical and/or drug claims are analyzed to determine if two claims indicating a member may have diabetes, and that are separated by at least three months, occur. If two claims meeting the criteria are identified, then the member is determined to have diabetes. For example, if two separate ICD codes in the 250 occur, separated by at least three months, or one such ICD code occurs and one drug code for anti diabetes medication occur, e.g., separated by at least three months, then the member may be determined to have diabetes.

Once the population is analyzed to establish who has diabetes, the historical health related characteristics of the diabetics are then used (analyzed) to establish a relationship between diabetes and the health related characteristics. For example, the health related characteristics are used to establish a neural network model, or regression model. The trained neural network and/or regression model will then be able to predict the likelihood a member of the population will acquire diabetes. In one embodiment, the neural network will also be able to establish who has, or may acquire, the related diabetic characteristics of metabolic syndrome and or glucose intolerance. Alternatively, these may be inputs to the neural network if available.

The established relationship may be reviewed to determine what the primordial characteristics associated with diabetes are. For example, it may be determined that salt intake, consumption of saturated fats, and alcohol consumption are three leading primordial characteristics of acquiring diabetes. In addition, it may be determined that smoking is not a primordial characteristic associated with diabetes. The population may then be reviewed using the established relationship. The health related characteristics of each member of the population not known to have diabetes may be analyzed using the relationship. The analysis may indicate the likelihood the person will acquire diabetes (e.g., 75% likely). In addition, the primordial characteristics associated with diabetes that are exhibited by the person may be identified. In this manner, the likelihood of the acquiring diabetes may be established along with what primordial characteristics are the primary contributors to this particular member having diabetes.

Once the populations health related characteristics are analyzed, the population may be ranked by the individual member's likelihood of acquiring diabetes. In this manner, the type of intervention may be recommended based on the risk of acquiring diabetes, and the primordial characteristics the member exhibits. In one embodiment, the interventions may be recommended by using another relationship (or an elaboration of the predictive relationship) to automatically make the recommendation based on the health related characteristics of the member, which may include the likelihood of acquiring diabetes and specific primordial characteristics exhibited, self-efficacy and readiness to change characteristics of the member, etc. In one embodiment, the intervention may include additional questionnaires or interviews to acquire more specific information associated with diabetes from the individual. Other forms of intervention include one on one counseling to convince the member of the seriousness of diabetes, the risk of acquiring diabetes associated with them, the ability to delay or prevent the onset of diabetes by changing specified lifestyle characteristics, and the specific actions the member may take to modify specific aspects of their lifestyle associated with the primordial characteristics. For example, if dietary issues are causing the member to be overweight, the intervention may include, suggested changes to dietary consumption, cookbooks directed towards the desired diet, or even corporate sponsored diet counseling or involvement in a commercial diet control program. The specific intervention recommended may be based on the likelihood of acquiring diabetes the person has, the members willingness to change their diet and belief that they will be successful in long term dietary change, and how much of a factor dietary issues were in establishing this particular members likelihood of acquiring diabetes.

Once the intervention recommendation is provided additional monitoring may occur to determine if the member followed through with the recommendation (including why they did or didn't follow through), whether the intervention helped reduce the targeted characteristic (e.g., the targeted primordial characteristic), and when the intervention did reduce the targeted characteristics, whether the ultimate occurrence of diabetes was either delayed (which may be a subjective determination) or prevented altogether. The results of this monitoring may then be used to update the established relationships. In addition, as incidents of diabetes occur, the health related characteristics of effected member may be used to further refine the established predictive relationship. In this manner, the health of the population may be analyzed and managed relative to diabetes.

Other aspects, objects, and advantages of the present invention can be obtained from a study of the drawings, the disclosure, and the claims.

What is claimed is:

1. A method of analyzing the health of a population, the population having a plurality of members, comprising the steps of:
establishing a plurality of health related characteristics associated with said population, said characteristics including a plurality of primordial characterstics;
establishing a relationship between said health related characteristics and at least one health condition; and
analyzing at least a portion of said population in response to said relationship.

2. A method, as set forth in claim 1, wherein the step of analyzing said at least a portion of said population further comprises the step of predicting a likelihood of at least one of said members developing said at least one health condition, in response to said relationship.

3. A method, as set forth in claim 1, wherein the step of analyzing said at least a portion of said population further comprises the step of predicting the likelihood of a member developing said at least one health condition in response to said relationship and said members health related characteristics.

4. A method, as set forth in claim 1, further comprising the step of identifying said primordial characteristics in response to said relationship.

5. A method, as set forth in claim 1, further comprising the step of establishing a prevalence of a health condition within said population in response to said health related characteristics.

6. A method, as set forth in claim 5, wherein the step of establishing said relationship further includes the step of establishing said relationship in response to said health related characteristics, said at least one health condition, and said prevalence.

7. A method, as set forth in claim 1, wherein the step of establishing a plurality of health related characteristics associated with said population further includes the step of establishing a plurality of self-reported characteristics associated with at least a portion of said population.

8. A method, as set forth in claim 5, wherein the step of establishing said prevalence further comprises the steps of:
establishing a plurality of claims associated with at least one os said members, said claims including at least one of a drug claim and a medical claim;
cross checking said plurality of claims; and
establishing said prevalence in response to said cross checked claims.

9. A method, as set forth in claim 8, wherein the step of cross checking includes the step of comparing two claims, said claims being separated by a predetermined period of time.

10. A method, as set forth in claim 1, further comprising the steps of:
establishing an incident of said disease associated with a member of said population;
establishing said plurality of characteristics associated with said member; and
modifying said relationship in response to at least said member's characteristics.

11. A method, as set forth in claim 3, wherein the step of predicting said member's likelihood of developing said condition further includes the step of establishing a stage of said condition in response to said prediction.

12. A method, as set forth in claim 3, wherein the step of predicting said member's likelihood of developing said condition further includes the step of predicting a time period associated with said development.

13. A method, as set forth in claim 3, further including the step of classifying said population in response to said prediction.

14. A method, as set forth in claim 3, further including the step of prioritizing said population in response to said prediction.

15. A method, as set forth in claim 3, wherein said primordial characteristic includes a disability characteristic, and further wherein said likelihood of development is predicted in response to said disability characteristic.

16. A method, as set forth in claim 3, further comprising the step of recommending an intervention in response to said predicted likelihood of development.

17. A method, as set forth in claim 16, further comprising the steps of:
establishing a plurality of intervention recommendations associated with said condition;
establishing a success characteristics of said recommended intervention;
establishing at least one of a readiness to change characteristic and a self-efficacy characteristic of said member; and
recommending said intervention in response to said plurality of intervention recommendations, associated intervention success characteristics, and member health related characteristics, said health characteristics including said self-efficacy and said readiness to change characteristic.

18. A method, as set forth in claim 16, further comprising the step of establishing a relationship configured to automatically recommend said intervention in response to said health related characteristics and said predicted likelihood of development.

19. A method, as set forth in claim 16, further comprising the step of monitoring an success characteristic of said intervention.

20. A method, as set forth in claim 19, further comprising the step of establishing a cause of said success characteristic.

21. A method as set forth in claim 1, further comprising the steps of:
determining at least one existing primordial characteristic associated with said condition and said member;
establishing an intervention associated with modifying said existing primordial characteristic;
establishing a success characteristic associated with said intervention.

22. A method of analyzing the health of a population, the population having a plurality of members; comprising the steps of:
establishing at least one primordial characteristic associated with a health condition;
applying an intervention in response to said characteristic;
monitoring a success characteristic of said intervention; and
determining a cause of said success characteristic.

23. A method, as set forth in claim 22, further comprising the step of establishing a prevalence of said condition within a population.

24. A method, as set forth in claim 23, wherein the step of establishing said prevalence further comprises the steps of:
establishing a plurality of claims associated with said member, said claims including at least one of a drug claim and a medical claim;
cross checking said plurality of claims; and
establishing said prevalence in response to said cross checked claims.

25. A method, as set forth in claim 24, wherein the step of cross checking includes the step of comparing two claims, said claims being separated be a predetermined period of time.

26. A method, as set forth in claim 25, wherein the step of establishing said characteristic further comprises the step of establishing said characteristic in response to said plurality of claims.

27. A method, as set forth in claim 26, further comprising the steps of:
establishing a plurality of self-reported data associated with at least a portion of said population having said condition.

28. A method, as set forth in claim 25, wherein said self-reported data includes at least one of a lifestyle characteristic, a family history characteristic, and a health characteristic.

29. A method, as set forth in claim 28, wherein the step of establishing said prevalence further includes the step of establishing said prevalence in response to said self-reported data.

30. A method, as set forth in claim 24, further comprising the step of establishing a likelihood of developing said condition.

31. A method, as set forth in claim 24, further comprising the step of establishing a predictive relationship between said primordial characteristics and said prevalence.

32. A method, as set forth in claim 31, further comprising the steps of:
applying said predictive relationship to at least a portion of said population; and
predicting a likelihood of developing said condition in response to application.

33. A method, as set forth in claim 30, wherein the step of applying said intervention further comprises the step of applying said intervention in response to said incidence prediction.

34. A method, as set forth in claim 30, wherein the step of establishing a predictive relationship further comprising the steps of:
establishing at least one objective of said relationship;
dynamically selecting a statistical analysis technique in response to said objective; and
establishing said relationship in response to said statistical analysis technique.

35. A method, as set forth in said 31, further comprising the steps of:
determining an incident of said condition;
collecting health related characteristics, at least a portion of which include said primordial characteristics, associated with said incident;
modifying said predictive relationship in response to said incident and said associated self-reported data.

36. A method, as set forth in claim 32, further comprising the step of establishing a predictive relationship between said primordial characteristics and said health related conditions.

37. A method, as set forth in claim 36, further comprising the steps of:
applying said predictive relationship to at least a portion of said population; and
predicting a likelihood of developing said condition in response to said application.

38. A method, as set forth in claim 37, wherein the step of applying said intervention further comprises the step of applying said intervention in response to said incidence prediction.

39. A method, as set forth in claim 22, further comprising the step of establishing a plurality of self-reported characteristics associated with said population.

40. A method, as set forth in claim 39, wherein the step of determining said cause of said effect further includes the step of determining said cause in response to said self-reported characteristics.

41. A method, as set forth in claim 40, wherein the step of determining said cause further includes the step of establishing a characteristic of self-efficacy in response to said self-reported data.

42. A method, as set forth in claim 41, further comprising the step of establishing said cause in response to said self-efficacy characteristic.

43. A method, as set forth in claim 42, further comprising the step of applying a second intervention in response to said effect.

44. A method, as set forth in claim 42, further comprising the step of modifying said intervention in response to said cause.

45. A system configured to analyze the health of a population, the population having multiple members, the system comprising;
a repository of health related characteristics associated with said population, said characteristics including a plurality of primordial characteristics; and
a controller configured to receive said health related characteristics, establish a relationship between said health related characteristics and at least one health condition, and analyzing at least a portion of said population in response to said relationship.
